(19) United States
(12) Patent Application Publication
(10) Pub. No.: US 2007/0212712 A1
(43) Pub. Date: Sep. 13, 2007

(54) Methods for identifying modulators of hedgehog autoprocessing

(76) Inventors: Xingbin Ai, Newton, MA (US); Charles Emerson, Winchester, MA (US); Henry Paulus, Boston, MA (US)

Correspondence Address: Kevin M. Farrell;Pierce Atwood, Suite 350, One New Hampshire Avenue, Portsmouth, NH, 03801, US

(21) Appl. No.: 11/634,300

(22) Filed: Dec. 5, 2006

Related U.S. Application Data
(60) Provisional application No. 60/742,472, filed on Dec. 5, 2005.

(51) Int. Cl. C12Q 1/68; C12Q 1/66; C12Q 1/37; C07F 9/80

(52) U.S. Cl. 435/6; 435/23; 435/8; 549/207

(57) ABSTRACT

The present invention relates to methods of screening for modulators of Hedgehog peptide autoprocessing with methods that are useful in both in vivo and high-throughput in vitro systems. The present invention also relates to reagents identified by the methods of the present invention for use in the modulation of Hh autoprocessing, especially as they relate to the treatment of illnesses and diseases that are caused, at least in part, by the misregulation of Hh autoprocessing.

### NOTICE OF FEDERAL FUNDING

This work was funded in part through grants from the National Institutes of Health (RO1 CA94089 and R43 AI53583). The government may have certain rights in this invention.

### BACKGROUND

Hedgehog peptides (Hh) are signaling ligands with key roles in stem cell specification and growth for tissue patterning in embryos and for stem cell renewal of adult tissues (reviewed by Ingham and McMahon, Genes Dev., 15:3059-3087, 2001; Beachy, et al., Nature, 432:324-31, 2004; U.S. Pat. Nos. 5,789,543 and 5,844,079 to Ingham, et al., incorporated herein by reference). Hh proteins are unique in vertebrates in that they are capable of autoprocessing, i.e., they are capable of self-cleavage thereby converting from a precursor peptide to an active mature peptide. Vertebrates have three Hh proteins, Sonic (Shh), Indian (Ihh) and Desert (Dhh) Hedgehogs. Hh activate the signaling in responsive stem cells by binding to the receptor Patched (Ptc), which relieves Ptc repression of Smoothened, a G-related protein, leading to the nuclear translocation of Gli transcriptional factors to activate and repress specific target genes controlling stem cell growth, apoptosis, differentiation and angiogenesis (Byrd, et al., Development, 129:361-72, 2002).

Hh have been identified as a tumor growth signal in a diversity of human cancers (Berman, etal., Nature, 425:846-851, 2003; Karhadkar, etal., Nature. 431:707-12, 2004). Tumors originating in esophagus, stomach, biliary tract, pancreas and prostate express high levels of Shh and Ihh, which stimulates the growth of stem cell-like tumor cells. Functional neutralization antibodies against Shh and Ihh have been shown to block the growth of these tumors in vitro and in xenografts, establishing that these tumors are completely dependent on Hh ligand for their growth. However, due to the bulky nature of antibodies, the use of antibodies intracellularly in organisms is problematic.

Since hundreds of thousands of people are diagnosed and die from cancer each year, is it critical that novel methods are developed for the detection of agents that can inhibit cancer growth. The identification of modulators (e.g., inhibitors) of Hh autoprocessing is a novel area ripe with possibility. There are no known prior art small molecule inhibitors for Hh autoprocessing. Moreover, there are not any known efficient methods or techniques leading to the development of an in vitro high-throughput (HTS) system for the identification of such inhibitors. Therefore, what is needed are techniques and methods for the efficient high-throughput screening of agent libraries (e.g., small molecule libraries) for the identification of molecules that are effective in the modulation of hedgehog autoprocessing.

### SUMMARY OF THE INVENTION

The present invention relates to methods for the identification of modulators of Hh autoprocessing. In a preferred embodiment, the invention relates to methods of identifying inhibitors of Hh autoprocessing utilizing in vitro cell-free or cell-based methods. In another aspect, the present invention relates to the screening of small molecule and combinatorial chemical libraries with the high-throughput, cell-free methods of the present invention. In another embodiment, the methods of the present invention are automated.

In other aspects, the present invention relates to the use of rigorous controls in said methods of high-throughput, cell-free screening to lessen false-positive and false-negative identifications of potential small molecule modulators of Hh autoprocessing. In yet another embodiment, the invention relates to using variations of the methods of the present invention in cell-based systems.

Hedgehog (Hh) autoprocessing plays an essential role in embryonic development (U.S. Pat. No.: 6,165,747 to Ingham, et al, incorporated herein by reference) and is involved in stem cell fate and the etiology of certain cancers. Examples are tumors of endodermal origin such as pancreatic, digestive tract and prostate cancers, etc. Hh autoprocessing may also play a role in fat absorption and be responsible for weight gain and hepatic steatosis in adults. The present invention relates to assays for the identification of reagents and agents that are effective in the modulation (i.e., the inhibition and promotion) of Hedgehog protein autoprocessing. One skilled in the art will realize the reagents and molecules identified by the methods of the present invention will be useful in the treatment of various illnesses and disease states related to the aberrant regulation of Hh autoprocessing such as tumors, angiogenesis, weight gain and loss and hepatic steatosis.

In another aspect of the invention, the reagents identified by the methods of the present invention as being effective in the modulation of Hh autoprocessing are also embodied in the presented invention for use in the modulation of Hh autoprocessing for the treatment, for example, of tumors, angiogenesis, weight gain and loss, hepatic steatosis, etc.

There are no known prior art organic small molecule modulators (i.e., inhibitors and promoters) for Hh autoprocessing. Moreover, there are no prior art protocols for an in vitro high-throughput (HTS) system for the identification of such Hh modulators. The following U.S. Patents by Beachy and coworkers have issued, however, and, although they relate to Hh and, in part, the modulation of Hh autoprocessing, none of the cited patents disclose high-throughput cell-free screening systems nor methods comprising rigorous control systems for the identification of modulators of Hh autoprocessing: U.S. Pat. Nos. 6,911,528, 6,867,216, 6,733,971, 6,432,970, 6,288,048, 6,281,332, 6,277,566, 6,214,794, 6,132,728 and 6,057,091 (all of which are incorporated herein by reference).

Unlike the present invention, U.S. Pat. No. 6,057,091 discloses the use of Hh fusion proteins for identifying autoprocessing inhibitors in cell-based systems with non-stringent control systems. Among the disclosures of the other patents are cell lines similar to those used in studying the inhibition of Hh processing in intact cells as well as upstream or downstream inhibitors of hedgehog signaling (affecting either cholesterol synthesis or downstream signaling steps but not Hh autoprocessing itself).

### DESCRIPTION OF THE FIGURES

FIG. 1 A shows the effects of intein inhibitor reagents on Shh signaling activity. Gli-luciferase reporter activity of 3T3 cells treated with 10 or 50 μmolar of each reagent, normalized to control cells treated with Shh alone, set at a value of 1. Reagents with no luciferase activity indicate cell toxicity. Reagents #1, 3, 4, 5, 7, 15, 16, 17 inhibited Shh signaling and were tested in autoprocessing independent signaling assays (B).

FIG. 1B shows the effects of selected intein inhibitors on autoprocessing-independent Shh-N signaling. Gli-luciferase activity induced by Shh-N dimethylsulfoxide (DMSO) compared with basal bovine serum albumin (BSA) control activity without Shh-N. 1 μM KAAD-cyclopamine (Cyc), a Smoothened inhibitor, inhibited Gli-luciferase activity in response to Shh-N activity, as did reagents #07, 15, 16, 17, identifying these reagents as non-specific inhibitors of Shh signaling. By contrast, reagents #3, 4, which strongly inhibited autoprocessing-dependent Shh activity (see A), only weakly inhibited Shh-N activity, identifying these as candidate autoprocessing inhibitors.

FIG. 2 shows a cell-based biochemical Shh autoprocessing assay to test the effects of selected intein reagents on Shh autoprocessing. Shh protein expressed in 3T3 cell line treated with control reagent #2 is efficiently self-spliced from a to, as assayed by Western blot analysis using antibodies that detect unprocessed 47 kD Shh precursor and processed 19 kD Shh-N product. Treatment of cells with reagents #3 and #4 inhibited Shh precursor processing. These biochemical data, together with cell-based Gli-luciferase reporter data (FIG. 1) identify intein inhibitors, #3 and #4, as candidate Hh autoprocessing inhibitors.

FIG. 3 shows the structure of reagent #4, an inhibitor of hedgehog auto-processing with a molecular weight of 313.

FIG. 4 shows the fluorescent assay for Hh autoprocessing based on the cholesterol-dependent autocleavage of an Hh-GFP fusion protein.

FIG. 5 shows an alternative assay for Hh autoprocessing based on fluorescence quenching of a GFP-Hedgehog-His₆ fusion and relieve of quenching upon cleavage.

FIG. 6 shows intein autoprocessing in vitro with the GFP/mini-intein fusion protein. Denatured inclusion bodies of GFP/mini-intein fusion protein were treated with 4,4′-dithiodipyridine, renatured by dialysis into buffer and incubated for 18 h at 25° C. in the absence or presence of TCEP. A) intein autoprocessing was followed by SDS-PAGE. B) Fluorescence generated by excitation at 395 nm.

FIG. 7 shows the structure of reagent #3, an inhibitor of hedgehog auto-processing with a molecular weight of 380.

### DETAILED DESCRIPTION OF THE INVENTION

Cell-free Assay Systems

In one aspect of the present invention, a method is contemplated for the screening of compounds that inhibit the autoprocessing of the Hedgehog protein in a cell-free system.

Such cell-free systems are composed of appropriate purified, genetically engineered fusion proteins to be used for various screening assays wherein test reagents are added to mixes of test and/or control peptides suitable for Hh autoprocessing. Such assays are conduced in a physiological environment such as a neutral pH range and physiological temperature. Detection can then be performed via an automated system (such as fluorescence-based plate readers) or by direct visual observation (for example, via the use of fluorescence detection or antibody binding and detection).

In a first compound screening embodiment of the present invention, the cell-free system includes a non-fluorescent or non-luminescent fusion protein, said fusion protein comprising: (1) a C-terminally truncated form of a protein which is fluorescent or luminescent in its full-length form; and (2) a Hedgehog autoprocessing domain fused in-frame to the C-terminal residue of the truncated form of the protein which is fluorescent or luminescent in its full-length form; wherein cleavage of the fusion protein by Hedgehog autoprocessing function results in fluorescence of luminescence of the C-terminally truncated protein.

The luminescent or fluorescent protein of the present invention may be selected from any number of reagents known to those practiced in the art. For example, the luminescent protein may be luciferase whereas the fluorescent protein may be selected, for example, from a group such as GFP, BFP, CFP and YFP and the red fluorescent protein from Discosoma and aequorin and other fluorescent molecules such as DsRed monomer, DsRed2, DsRed-express, AsRd2, HcRed1, AmCyan1, ZsYellow1, and ZsGreen1from Clontech, Mountain View, Calif. or similar products from Evrogen, Moscow, Russia; Invitrogen, Carlsbad, Calif., etc.

In this first compound screening embodiment, the cell-free system also includes a nucleophile initiator. A nucleophile is a reagent that is attracted to centers of positive charge. A nucleophile participates in a chemical reaction by donating electrons to a species known as an electrophile in order to form a chemical bond. In the context of the present invention, a nucleophile initiator is a reagent that serves to initiate the autoprocessing reaction of the Hedgehog protein. In a preferred embodiment, the nucleophile initiator is a sterol (e.g., cholesterol, desmosterol, β-sitosterol, 7β-hydroxycholesterol, ergosterol, or 7-dehydrocholesterol) or a thiol.

Also provided in connection with this embodiment is a compound to be tested for the ability to inhibit (either partially or totally) Hedgehog autoprocessing. The test reagents of the present invention may be selected from small molecule libraries and other libraries including combinatorial chemical libraries. Such libraries are known in the art and are available commercially. Additionally, proprietary libraries are also available for use from collaborators and others. Additionally, the synthesis and screening of small molecule libraries (e.g., combinatorial chemical libraries) are well known in the art (See, for example, U.S. Pat. No. 6,060,596 to Lerner; U.S. Pat. No. 6,185,506 to Cramer, et al.; U.S. Pat. No. 6,377,895 to Horlbeck; U.S. Pat. No. 6,936,477 to Still, et al.; Shipps, et al., Proc. Natl. Acad. Sci. USA, 94:11833-11838, 1997; Stockwell, et al., Chemistry & Biology, 6:71-83, 1999, all of which are incorporated herein by reference). (See, also, for example, www.combichem.net; www.combichemistry.com; www.combinatorial.com and pubs.acs.org/journals/jcchff/).

Candidate reagents encompass numerous chemical classes, though typically they are organic molecules, preferably small organic compounds having a molecular weight of more than 50 and less than about 3,500 daltons. Candidate reagents comprise functional groups necessary for structural interaction with proteins, particularly hydrogen bonding and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Non-limiting examples of suitable small molecule libraries are given in Example 5.

Candidate reagents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The contemplated method comprises forming an incubation mixture including the compositions recited above (i.e., the cell-free system, the fusion protein, the nucleophile initiator and the test reagent), incubating the mixture under conditions and for a period of time appropriate for Hedgehog autoprocessing, detecting fluorescence or luminescence in the incubation mixture and comparing the fluorescence or luminescence detected to fluorescence or luminescence detected using an otherwise identical incubation mixture which lacks the test reagent, a decrease in fluorescence or luminescence being indicative of the presence of a compound that inhibits autoprocessing of Hedgehog. Times and conditions appropriate for Hedgehog autoprocessing are typically about 3 hours at about 37° C. and pH 7. However, these conditions are not critical. For example, the temperature can be varied between about 20° C. and 40° C. and the pH from about 6.5 to 8.0.

The detection of luminescence or fluorescence as the result of Hedgehog autoprocessing may be performed by, for example, spectroscopy, automated plate readers, fluorescent (or luminescent) microscopy, etc. Such techniques are well known to those practiced in the art.

Another aspect of the present invention relates to second compound screening assay that is carried out in a cell-free system. In connection with this method, provided are a fusion protein consisting of a Hedgehog protein in which a large portion or most of the N-terminal signaling domains replaced by a small peptide that can be selectively fluorescently labeled. Following the selective labeling, the cleavage of the fusion protein by the Hedgehog autoprocessing function releases the fluorescently labeled small peptide thereby resulting in a detectable change in fluorescence polarization. The small peptide, which can be selectively labeled can be fused to the truncated Hedgehog protein without impairing cholesterol-dependent autoprocessing (Porter, et al., Nature, 374:363-366, 1995).

The method comprises, for example, forming a cell-free incubation mixture comprising the Hedgehog fusion construct as described above, a weakly or non-fluorescent reagent for the fluorescent labeling of the fusion protein, a nucleophile initiator and a compound to be tested for the ability to inhibit (partially or totally) Hedgehog autoprocessing. This mixture is incubated under conditions and for a period of time appropriate for Hedgehog autoprocessing. Fluorescence polarization in the mixture is detected and compared with fluorescence polarization detected in an otherwise identical incubation mixture that lacks the test compound. A decrease in fluorescence polarization in the mixture with the test compound relative to that determined for the otherwise identical incubation mixture which lacks the test compound, is indicative of the ability of the test compound to inhibit autoprocessing of Hedgehog protein.

As mentioned previously, the N-terminal Hedgehog signaling domain is replaced by a small polypeptide without impairing cholesterol-dependent autoprocessing (See, for example, Porter, et al., Nature, 374:363-366, 1995, which is incorporated herein by reference). Since the C-terminal domain of the Hedgehog peptide is the autoprocessing domain, the fusion of a tag on the N-terminal does not affect autoprocessing. A preferred peptide includes the sequence Cys-Cys-X-X-Cys-Cys [SEQ ID NO.: 1], where X is any amino acid. In a preferred embodiment, X-X are the amino acids Gly-Pro giving the sequence Cys-Cys-Gly-Pro-Cys-Cys [SEQ ID NO.: 2]. Such sequences are rare in cells or cell-free systems unless added. These sequences bind to a fluorescent biarsenical ligand with a nanomolar or lower dissociation constant. Biarsenical molecules are weakly fluorescent dyes that bind extremely tightly to very small peptide motifs containing four cysteines, which can be genetically incorporated into proteins, and upon binding become intensely fluorescent. The dyes readily cross biological membranes and are relatively nonfluorescent until they find the target peptide sequences. An example of a suitable fluorescent biarsenical is 4′,5′-bis(1,3,2-thothioarsolan-2-yl)fluorescein, which, upon binding to -Cys-Cys-X-X-Cys-Cys-, yields intense fluorescence emission at 535 nm upon excitation at 480 nm, whereas the free biarsenical reagent has a 50,000-fold lower fluorescence. Numerous other fluorescent biarsenical molecules as well as biarsenical molecules that may be conjugated to fluorescent moieties are known in the art (for example, see, Zhang, et al., Nature Review Molecular Biology, 3:906-918, 2002; and U.S. Pat. Nos. 5,932,474 and 5,998,204 to Tsein, incorporated herein by reference). Upon autoprocessing, either in the presence of cholesterol (or other sterol) or an excess of a thiol (other than a dithiol such as DTT), the approximately 30 kDa fluorescent-tagged fusion protein will be cleaved to an approximately 3 kDa fluorescent peptide, a change that can easily be measured by a decrease in fluorescence polarization.

Briefly, the concept of molecular movement and rotation is the basis of fluorescence polarization. Fluorescence polarization is defined by the following equation:
P=(V−H)/(V+2H)
where P equals polarization, V equals the vertical component of the emitted light, and H equals the horizontal component of the emitted light of a fluorophore when excited by vertical plane polarized light. As can be seen from the equation, P (“polarization unit”) is a dimensionless entity and is not dependent on the intensity of the emitted light or on the concentration of the fluorophore. This is the fundamental power of FP. Thus, upon autoprocessing of the Hedgehog peptide, the fluorescence polarization of the molecule, as determined by observing the molecule when exposed to polarized light, will be lower because the molecule will rotate faster when released from the larger Hedgehog peptide. The differences in observable fluorescent polarization can, therefore, be used to measure Hedgehog autoprocessing.

Thus, in the present instance, cleavage of the small fluorescent peptide fused to Hedgehog, upon autoprocessing, will result in a small, tagged, molecule, which rapidly tumbles out of the plane of polarization. In contrast, uncleaved Hedgehog, which rotates at a slower rate compared to the cleaved products, will emit a larger amount of energy in the same plane as the excitation energy. Thus, a determination of the activity and rate of the Hedgehog's ability to autoprocess is detectable by measuring the polarization signal with a decrease in the polarization signal (i.e., a decrease in fluorescence polarization) indicative of autoprocessing taking place.

Cell-based Assay Systems

In another aspect of the present invention, a method is contemplated for the screening of compounds that inhibit the autoprocessing of the Hedgehog protein in cell-based systems. In one aspect, the invention utilizes a test cell comprising a first expression vector encoding a functionally active, unprocessed Hedgehog protein and a second expression vector comprising a nucleotide sequence encoding a functional luciferase protein driven by 8 tandem repeats of Gli-binding sequences, or regulatory sequences of other Hedgehog-responsive genes (e.g., Gli, Glil, MIM/BEG4, patched, N-Myc, IGF2). The construction of expression vectors is well known in the art as is exemplified by the teachings in Sambrook, J., Fritsch, E. F., and Maniatis, T., Molecular Cloning: A Laboratory Manual. Cold Spring Harbor Laboratory Press, N.Y., Vol. 1, 2, 3 (1989), which is herein incorporated by reference. Suitable expression vectors for the present invention would include, for example, Bluescript™, or any of the Flexi200 Vector systems from Promega (Madison, Wis.).

Luciferase-based assays (both single and dual luciferase assay systems) and variations thereof, all of which are known to those skilled in the art, may be used in the present invention. In the luciferase-based assay, the detection of Hedgehog autoprocessing is via the assays the luciferase expression in response to Hedgehog activity. Luciferase luminescence may be detected, for example, with microscopy, scintillation counters or with the use of automated plate readers. Once Hedgehog is activated by autoprocessing, it is secreted and activates signaling in responsive cells by binding to the receptor Patched (Ptc), which relieves Ptc repression of Smoothened, a G-related protein, leading to the nuclear translocation of Gli transcriptional factors to activate the luciferase gene expression. In the present assay system, the same cells may be transfected with both Hedgehog and with the Hedgehog-responsive luciferase gene thereby allowing for a more efficient assay system.

Such cell-based assays would require the transfection of the expression vectors of the present invention into cells suitable for the assay. Suitable transfection methods include DEAE-dextran, calcium phosphate precipitation, Lipofectamine™ (Invitrogen, Carlsbad, Calif.), Profectin™ (Promega, Madison, Wis.) and other liposome methods, direct microinjection, electroporation and bolistic particle delivery, for example. Any cell line may be used for the present invention although the preferred cell line is the 3T3 mouse cell line.

After expression of the protein had been ascertained (e.g., by Western blotting, cell sorting, immunoflourescence or historical reference), a test compound is added to the transfected cells and the mixture is incubated under conditions and for a period of time appropriate for Hedgehog autoprocessing. Luminescence is then detected in the incubation mixture and compared to the luminescence of an otherwise identical incubation mixture that lacks the test compound, a decrease in luminescence of the incubation mixture being indicative of the presence of a test compound that inhibits autoprocessing of Hedgehog.

Another aspect of the present invention is a cell-based system for the screening of Hedgehog inhibiting compounds wherein a test cell has been transfected with an expression vector encoding a full-length, unprocessed Hedgehog protein and the test cell is contacted with a test compound under conditions and for a period of time appropriate for Hedgehog autoprocessing. The efficiency of Hedgehog processing is then detected by Western blotting. In brief, Western blotting is performed, for example, as follows: Proteins are separated using SDS-polyacrylamide gel electrophoresis (SDS-PAGE). They are then transferred to a (for example) nitrocellulose membrane. The nitrocellulose is blocked with a blocking agent (e.g., milk). The nitrocellulose membrane is incubated with, first, a primary antibody and, then, with a secondary antibody. The secondary antibody should be an antibody-enzyme conjugate. The labeled secondary antibody is then detected with, for example, x-ray film, phosphor imager or other detection device. The efficiency of Hedgehog autoprocessing is than compared to a otherwise identical mixture that lacks the test compound, wherein a lower efficiency of Hedgehog protein autoprocessing in the incubation mixture, relative to that determined using the otherwise identical incubation mixture, is indicative of the ability of the test compound of step to inhibit Hedgehog protein autoprocessing.

The assay described above may also be performed by using mass spectrometry to detect Hedgehog autoprocessing instead of, or in addition to, Western blotting. In brief, mass spectrometry is performed, for example, as follows: In MALDI-TOF mass spectrometry, a substance is deposited on a matrix and bombarded with a laser beam having sufficient energy to volatilize and impart a charge on the molecule. The charged molecules are accelerated in a vacuum through a magnetic field and are sorted on the basis of mass-to-charge ratio. Since the bulk of the ions produced in the mass spectrometer carry a unit charge, the value m/e is equivalent to the molecular weight of the fragment.

Another aspect of the present invention is a cell-based system for testing the specificity of Hedgehog inhibiting compounds comprising both a test cell and a control cell. The test cell comprises two expression vectors, the first expression vector comprising a nucleotide sequence encoding a functionally active, unprocessed Hedgehog protein; and the second expression vector comprising a nucleotide sequence encoding a functional luciferase protein driven by 8 tandem repeats of Gli-binding sequences. The control cell comprises two expression vectors, the first expression vector comprising a nucleotide sequence encoding a pre-processed Hedgehog protein the second expression vector comprising a nucleotide sequence encoding a functional luciferase protein driven by 8 tandem repeats of Gli-binding sequences. After expression of the protein had been ascertained (e.g., by Western blotting, cell sorting, immunofluorescence or historical reference), a test compound is added to the transfected test cells and control cell. A test incubation mixture is made by contacting the test cells with a test compound and a control incubation mixture is made by contacting the control cells with the same test compound, both incubation mixtures under conditions and for a period of time appropriate for Hedgehog autoprocessing. Luminescence is then detected in the test incubation mixture which has the expression vector encoding the unprocessed Hedgehog protein and compared to the luminescence of the control incubation mixture that has the expression vector encoding the preprocessed Hedgehog protein, a decrease in luminescence of the incubation mixture being indicative of the presence of a test compound that inhibits autoprocessing of Hedgehog.

Another cell-based assay for Hedgehog autoprocessing inhibitors is to analyze the gene expression of Hedgehog-responsive gene (also known as “gene transactivity” since the activation of one gene induces the activation of another gene) (e.g., Gli, Glil, MIM/BEG4, patched, N-Myc, IGF2). The test cell comprises the expression vector comprising a nucleotide sequence encoding a full-length Hedgehog protein. After expression of the protein had been ascertained (e.g., by Western blotting, cell sorting, immunofluorescence or historical reference), a test compound is added to the transfected cells and the mixture is incubated under conditions and for a period of time appropriate for Hedgehog autoprocessing. The expression of Hedgehog-responsive genes will be assayed by RT-PCR or Western blot and compared between incubation mixture and an otherwise identical incubation mixture that lacks the test compound, a decrease in Hedgehog-responsive gene expression being indicative of the presence of a test compound that inhibits autoprocessing of Hedgehog.

Intein Autoprocessing Inhibitors

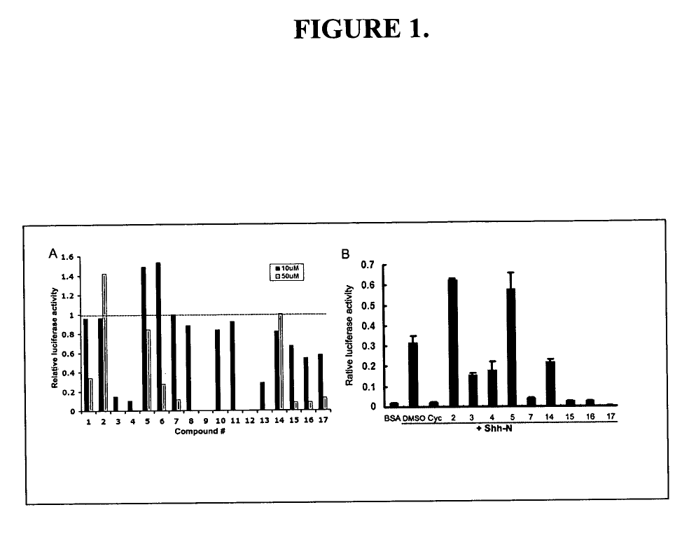

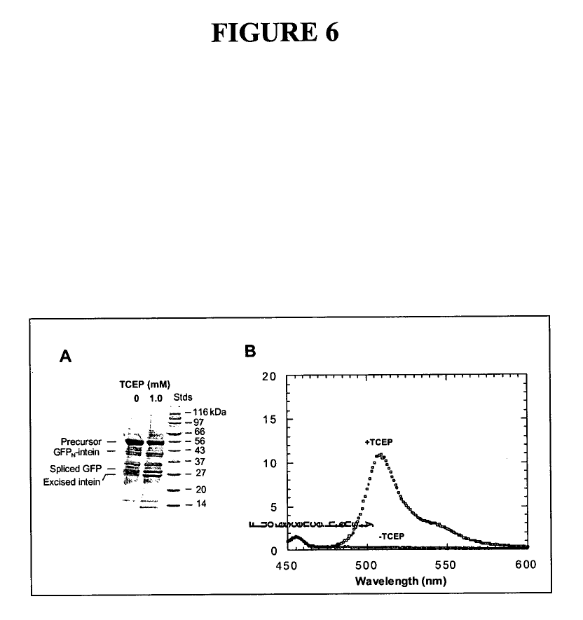

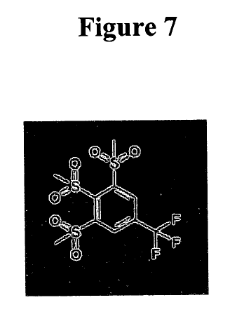

Furthermore, another aspect of the present invention relates to the compounds identified as inhibitors of intein autoprocessing by the method described in Example 5 and illustrated in FIG. 6. Owing to the great similarity of inteins and hedgehog autoprocessing domains, inteins cans serve as surrogates of hedgehog proteins in the screening for hedgehog autoprocessing inhibitors, a small but significant fraction of intein inhibitors being able also to inhibit hedgehog autoprocessing. Non-limiting examples of such compounds are compound #4 of FIG. 1 and compound #3 of FIG. 7.

Hedgehog Molecular Biology

Although the present invention is not limited to any particular theory, it is believed that the following discussion of Hedgehog peptides (Hh) will aid in the understanding of the invention. Hedgehog peptides are signaling ligands with key roles in stem cell specification and growth for tissue patterning in embryos and for stem cell renewal of adult tissues (reviewed by Ingham and McMahon, Genes Dev., 15:3059-3087, 2001; Beachy, et al., Nature, 432:324-331, 2004). Vertebrates have three Hh proteins, Sonic (Shh), Indian (Ihh) and Desert (Dhh) Hedgehogs. Hh activate signaling in responsive stem cells by binding to the receptor Patched (Ptc), which relieves Ptc repression of Smoothened, a G-related protein, leading to the nuclear translocation of Gli transcriptional factors to activate and repress specific target genes controlling stem cell growth, apoptosis and differentiation.

Hh have been identified as a tumor growth signal in a diversity of human cancers (Berman, et al., Nature, 425:846-851, 2003; Karhadkar, et al., Nature, 431:707-712, 2004). Tumors originating in esophagus, stomach, biliary tract, pancreas and prostate express high levels of Shh and Ihh, which stimulates the growth of stem cell-like tumor cells. Functional neutralization antibodies against Shh and lhh have been shown to block the growth of these tumors in vitro and in xenografts, establishing that these tumors are completely dependent on Hh ligand for their growth. As such, the present invention, as detailed above and in the Experimental section, relates to methods for the identification of small molecule drugs that inhibit the autoprocessing of Hh proteins, a process that is essential for the production and functional activation of Hh proteins and is unique to Hh among all vertebrate (e.g., human) known proteins. Inhibitors of Hh autoprocessing, therefore, should provide highly specific and effective drugs to block the growth of ligand-dependent (for example) Hh tumors.

The regulation of Hh autoprocessing has also been shown to be necessary for both weight regulation (Buhman, et al., J. Nutr., 134:2979-84, 2004) and stem cell differentiation (Ahn and Joyner, Nature 437:894-897, 2005). Thus, it is one aspect of the invention that the compounds identified as regulators (modulators) of Hh autoprocessing by the methods of the present invention are useful for both the regulation of weight and stem cell differentiation in animals.

Hedgehog are the only known self-spliced proteins in vertebrates. All three Hh proteins are expressed as a ˜45 kDa precursor, which undergoes autoprocessing to generate a secreted amino-terminal fragment Hh-N and a carboxyl-terminal fragment Hh-C. Hh-N, which is produced with a cholesterol moiety covalently attached to its carboxyl terminus, is the functional signaling ligand, and Hh-C is the auto-processing domain that is responsible for peptide bond cleavage and cholesterol transfer. Previous crystallographic study and in vitro splicing assays have demonstrated that Drosophila Hh-C not only shares highly similar sequence homology and crystal structure with the autoprocessed regions of inteins, Hh-C also utilizes highly similar reaction mechanisms as inteins in autoprocessing, which include an initial conversion of a peptide bond to a thioester linkage and subsequent attack by nucleophilic groups (Hall, et al., Cell, 91:85-97, 1997). However, in contrast to other autoprocessed proteins, Hh autoprocessing involves attack on the thioester group by cholesterol rather than by a Cys/Ser/Thr residue at the intein/C-extein junction (Hall, et al., Cell, 91:85-97, 1997; Paulus, Annu. Rev. Biochem., 69:447-496, 2000). All three human Hh contain conserved amino acid residues required for autoprocessing. Recently, as exemplified below, it has been shown human Shh-C also displays autoprocessing activities in vitro. Therefore, human Hh precursors undergo similar, but not identical, autoprocessing process to generate functional Hh-N signaling ligands.

### EXPERIMENTAL

### Example 1

### Luciferase-based Assay for the Screening for Hedgehog Autoprocessing Inhibitors

For this project, a directed biochemical screen to identify small molecule inhibitors of Hh autoprocessing as drugs for treatment of, for example, ligand-dependent Hh cancers, was constructed.

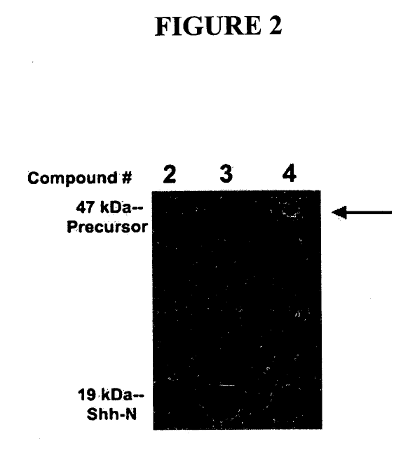

The primary and 3-D structures of inteins and the Hh autoprocessed domain are highly similar and the first step of protein splicing and Hh autoprocessing are identical (Paulus, Annu Rev Biochem 69:447-496, 2000). These similarities imply that there should be a class of protein splicing inhibitors that also inhibit Hh autoprocessing. Since our high-throughput screen for protein splicing inhibitors has led to a large collection of reagents, it was worthwhile to test these for anti-Hh activity. Indeed, of the 41 protein splicing inhibitors tested in a cell-based assay for Hh autoprocessing, two were found also to inhibit Hh autoprocessing (see FIGS. 1 and 2). This constitutes proof-of-concept that small molecules can inhibit Hh autoprocessing and suggests a method for obtaining such inhibitors.

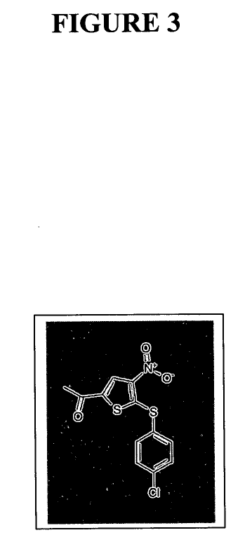

This cell-based assay (FIGS. 1A and 1B) made use of Hh-responsive 3T3 mouse cells stably transfected with a Shh plasmid expressing full length Shh protein that undergoes autoprocessing to produce functionally active Shh protein. Cells incubated with pre-spliced N-Shh protein were used as a control. Shh signaling was assayed in control and treated cells using with dual luciferase reporter assay of luciferase activity expressed from an integrated copy of a Gli-luciferase reporter gene linked to of 8 tandem repeats of Gli transcription factor-binding sites, normalized to activity of a Renilla gene under the control of minimum TK promoter, to monitor toxicity and cell survival. Inhibition was confirmed by Western blot analysis of Hh autoprocessing (FIG. 2). The structure of the most active inhibitor, reagent #4, is shown in FIG. 3. The structure of another active inhibitor, reagent #3, is shown in FIG. 7. Additional cell-based assays for testing the activities of candidate Hh autoprocessing inhibitors in blocking full-length Hh signaling include the measurement of Hh target gene expression (Patched, Gli , IGF2, N-myc; for example, via a luciferase-based assay) and chondrocyte differentiation of 10T½ cells. Persons skilled in the art will note that the above assays are also suitable for use in a cell-free assay system.

### Example 2

### GFP Based Assay for the Screening for Modulators of Hedgehog Autoprocessing

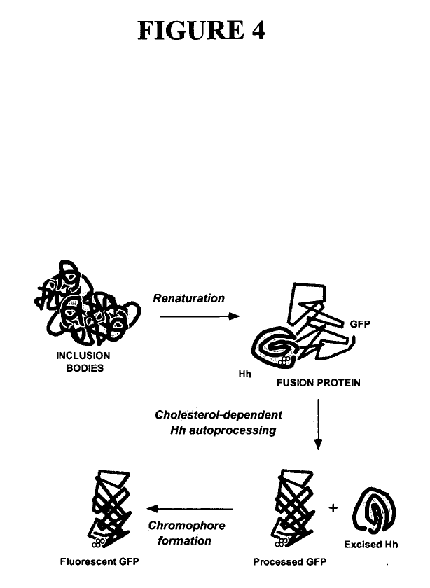

This high-throughput assay, summarized in FIG. 4, involves the fusion of the Hh autoprocessing domain to the C-terminus of a slightly truncated GFP molecule in a manner that blocks chromophore forrnation but allows it to occur when the bulky Hh autoprocessing domain is replaced by cholesterol. It takes advantage of the following pieces of information: (1) Truncated GFPI₁₋₂₂₄ is incapable of chromophore formation, but can do so when complemented by GFP₂₂₅₋₂₃₀.(Cabantous, et al., Nature Biotech 23:102-107, 2005); (2) GFP₁₋₂₂₅ is incapable of chromophore formation but GFP₁₋₂₃₂ is fully fluorescent (Dopf and Horiagon, Gene 173:39-44, 1996); and (3) the crystal structure of GFP (1GFL) shows that residues 230-238 are disordered (Tsien, Annu Rev Biochem 67:509-544, 1998). This means that the insertion of the Hh autoprocessing domain downstream of GFP residue 230 would have little effect on chromophore formation (although there may be some steric blockage due to fusion to a bulky protein—note that most GFP fusion proteins employ a relatively long linker between the GFP C-terminus and the fusion partner), but insertion at a site between residues 224 and 230will definitely block GFP fluorescence. Because the globular Hh autoprocessing domain is more bulky than cholesterol, there will be an insertion site at which the removal of Hh by cholesterol-dependent autoprocessing will allow chromophore formation.

GFPuv, the same form used for our intein constructs (Gangopadhyay, et al., Anal Chem 75:2456-2462, 2003), will be fused to the Hh autoprocessing domain, at residues between 224 through 232. The initial choice is insertion downstream of Gly228 or Gly232, which allows optimal autoprocessing, but other sites (i.e., downstream of Val224, Thr225, Ala226, Ala227, Gly228, Ile229, Thr230, or His231, for example) are also suitable as insertion sites for producing the desired results. These fusions are easily constructed by “gene SOEing (splicing by overlap extension)” (Horton, et al., Biotechniques 8:528-535, 1990). At 37° C. the fusion proteins are expressed as inclusion bodies and are denatured and then renatured at lower temperatures allowing a certain fraction of soluble fusion proteins will be produced. Persons skilled in the art will see that multiple variations of the denature-renature process will produce suitable fusion proteins. The fusion proteins of choice are non-fluorescent. These are monitored for gain of fluorescence upon Hh autoprocessing either in the presence of 20 mM DTT (dithiothreitol; which leads to the removal of the Hh domain) or cholesterol (which leads to the replacement of the Hh domain by cholesterol). The non-fluorescent constructs of choice are those that achieve fluorescence upon cholesterol-induced autoprocessing.

### Example 3

### FRET Based Assay for the Screening for Modulators of Hedgehog Autoprocessing

It is also worthwhile to focus on constructs that fluoresce even in the absence of autoprocessing as models for optimizing the autoprocessing conditions of GFP-Hh fusion proteins. Indeed, a fluorescent GFP-Hh fusion protein, in which the Hh autoprocessing domain is fused to GFP downstream of residue 252, can be the basis for an alternate, FRET-(fluorescence resonance energy transfer) based high throughput screening system, which is an embodiment of the present invention.

This type of assay is based on the observation that the NTA-Rhodamine derivative (NTA-I) in metal ion complex with an N-terminal His-tag almost completely quenches GFP fluorescence (Guignet, et al., Nature Biotechnology 22:440-444, 2004). Since the N- and C-termini of the Hh autoprocessing domain are in close proximity, a C-terminal His-tag of a GFP-Hh fusion protein will show a similar relationship to the GFP chromophore as an N-terminal GFP His-tag and NTA-I complexed to that C-terminal His-tag of the GFP-Hh fusion protein causes almost complete quenching of GFP fluorescence.

However, upon autocleavage, the covalent link between GFP and Hh is broken and fluorescence quenching is relieved. Thus, Hh autoprocessing leads to a substantial increase in GFP fluorescence (about 20-fold), thereby providing for a sensitive assay.

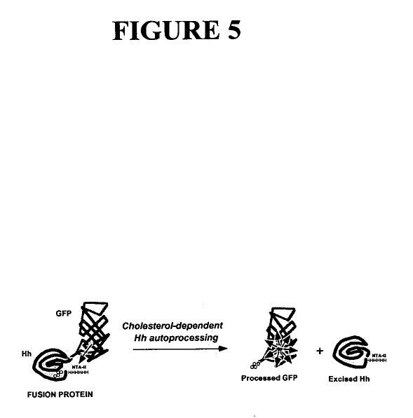

The assay (FIG. 5) involves a GFP-Hh fusion with Hh being fused to the C-terminus of GFP, so as not to interfere with fluorophore formation, and with a His-tag at the Hh C-terninus. (In case a C-terrninal His-tag interferes with autoprocessing, a short spacer is used between the C-terminus and the His-tag.) After confirming by SDS-PAGE that thiol-dependent and cholesterol-dependent cleavage occurs, the reaction is carried out in the presence of, for example, 20 μM NTA-I, which quenches the fluorescence of the fusion protein by about 95%. The fluorescence quenching is completely overcome by autocleavage, with a signal-to-noise ratio of about 10 upon 50% autoprocessing. This allows the reliable detection of reagents that produce 50% or greater inhibition.

This assay is considerably simpler in its structural requirements than the assay in Example 2 and does not involve GFP chromophore formation as a secondary reaction that can be inhibited by in a separate reaction unrelated to the autoprocessing step, but suffers from the disadvantage that the signal-to-noise ratio is less.

Similar assays as the one described above are based on other fluorescent proteins such as the GFP variants BFP, CFP and YFP, the red fluorescent protein from Discosoma and aequorin or based on luciferase proteins such as the firefly and Renilla luciferase. The principles of all these assays is basically the same: The fusion of the hedgehog autoprocessing domain to an amino acid residue near the C-termninus of the fluorescent protein or the luciferase in such a manner that the presence of the hedgehog protein blocks the fluorescence or luciferase function but that the replacement of the bulky hedgehog protein by cholesterol as a result of autoprocessing restores the ability of the protein to fluoresce or omit light. Since the atomic structures of all of these proteins are known, the selection of an appropriate fusion site for the hedgehog autoprocessing domain is a relatively straightforward matter based on the teachings contained herein.

### Example 4

### High Throughput Hedgehog Processing Assay Based on Fluorescence Polarization

An altogether different type of Hedgehog processing assay takes advantage of the fact that the N-terminal Hedgehog signaling domain can be replaced by a small polypeptide without impairing cholesterol-dependent autoprocessing (Porter, et al., Nature, 374:363-366, 1995). Replacing the N-terminal signaling domain with a peptide that selectively binds a fluorescent moiety allows a change in fluorescent polarization following autoprocessing by Hedgehog. There are many ways in which such N-terminal labeling can be effected. Perhaps the simplest approach involves the introduction of an affinity tag in the N-terminal domain to which a fluorescent probe can selectively bind with high affinity. One such labeling approach involves the use of Flash-tags (Griffin, et al., Science, 281:269-272 1998 and U.S. Pat. Nos. 5,932,474; 6,008,378; 6,054,271; 6,451,569 and 6,686,458, all to Tsien and Griffin, all of which are incorporated herein by reference). This approach can easily be implemented by expressing a fusion protein in which the N-terminal signaling domain of a functional Hedgehog protein is replaced by a small polypeptide that includes the sequence Cys-Cys-X-X-Cys-Cys [SEQ ID NO.: 1], where X is any amino acid. In a preferred embodiment, X-X are the amino acids Gly-Pro giving the sequence Cys-Cys-Gly-Pro-Cys-Cys [SEQ ID NO.: 2]. This peptide selectively binds a fluorescent biarsenical such as 4′,5′-bis(1,3,2-dithioarsolan-2-yl)fluorescein to yield intense fluorescence at 535 nm emission upon excitation at 480 nm, whereas the free biarsenical reagent has a 50,000-fold lower fluorescence. Upon autoprocessing, either in the presence of cholesterol or an excess of a thiol other than a dithiol such as DTT, the approximately 30 kDa fluorescent protein is cleaved to an approximately 3 kDa fluorescent peptide, a change that can easily be measured by fluorescence polarization. The assay for the inhibition of Hedgehog autoprocessing inhibitors therefore involves treating the Flash-tagged Hedgehog autoprocessing domain with the fluorescent biarsenical and comparing the fluorescence polarization signal in the absence and the presence of inhibitor candidates. Inhibition is indicated by a reduction in the fluorescence polarization signal. A similar assay for Hedgehog autoprocessing is implemented with fluorescent fusion proteins in which the N-terminal moiety is fluorescently labeled by other methods, a technique that could be practiced by one skilled in the art with the teachings contained herein.

### Example 5

### Enrichment of Compound Libraries for Hedgehog Autoprocessing Inhibitors by Screening for Protein Splicing Inhibitors

The fact that both the primary and 3-D structures of inteins and the Hh autoprocessing domain are highly similar and that the first step of protein splicing and Hh autoprocessing are identical (Paulus, Annu. Rev. Biochem., 69:447-496, 2000) implies that there should be a class of protein splicing inhibitors that also inhibits Hh autoprocessing. This screening system makes use of GFP as a fluorescent indicator and is based on the observation that GFP, interrupted by a Hh autoprocessing site, can undergo splicing to produce a functional protein (Ozawa, et al., Anal Chem 72:5151-5157, 2000). The coding sequence for a RecA mini-intein derived from Mycobacterium tuberculosis was inserted adjacent to the codon of residue 129 in the gene of GFPuv to yield a hybrid plasmid. In E. coli JM109(DE3), the GFP-RecA mini-intein fusion protein is expressed as inclusion bodies and is solubilized in, for example, 8 M urea and purified as a non-fluorescent protein, which is stabilized to allow prolonged storage by blocking the thiol groups with 4,4′-dithiopyridine. Upon renaturation by dilution and dialysis and reduction of the disulfide bonds with TCEP, autoprocessing is observed, accompanied by the appearance of fluorescence, as shown in FIG. 6 (Gangopadhyay et al., 2003). This provides a sensitive assay for Hh autoprocessing with virtually no background, which is adapted to a 384-well format to screen for Hh autoprocessing inhibitors. The signal-to-noise ratio under high-throughput conditions is about 20, with a Z-factor (Zhang, et al., J Biomol Screen, 4:62-73, 1999) of 0.88.

The 384-well assay was used to screen about 80,000 reagents from diverse libraries, utilizing the facilities of the Institute of Chemistry and Cell Biology (ICCB) of Harvard University. The reagent library screening will involve, for example, the following libraries: Commercial Diversity Set (5,056 reagents), Diversity Oriented Synthesis (16,030 reagents), ICCB Bioactives (489 reagents), ChemDiv Combilab/Intemational (28,864 reagents), Bionet (6,168 reagents), Maybridge (16,807 reagents), Peakdale (3,168 reagents) and Mixed Commercial Plates (1,254 reagents).

Secondary screening of the hits to eliminate inhibitors of GFP chromophore formation and reagents with IC₅₀ values greater than 40 μM are reagents were purchased and studied in more detail. Sixty compounds that inhibited the RecA intein in the low micromolar range were tested for inhibition of hedgehog autoprocessing in the cell-based assay systems described in Example 1 and two compounds were found (Reagents #3 and #4) that significantly inhibited hedgehog autoprocessing. This demonstrates that selecting intein inhibitors from compound libraries can serve as a method of enrichment of compounds that inhibit hedgehog autoprocessing.

We claim:

1. A method for the screening of compounds that inhibit the autoprocessing of the Hedgehog protein in a cell-free system, said method comprising:
a) providing:
i) an expression vector encoding a non-fluorescent or non-luminescent fusion protein, said fusion protein comprising: 1) a C-terminally truncated form of a protein which is fluorescent or luminescent in its full-length form; and 2) a Hh autoprocessing domain fused in-frame to the C-terminal residue of the truncated form of the protein which is fluorescent or luminescent in its full-length form; wherein cleavage of the fusion protein by Hh autoprocessing function results in fluorescence of luminescence of the C-terminally truncated protein,
ii) a nucleophile initiator; and,
iii) a compound to be tested for the ability to inhibit Hedgehog autoprocessing;
b) forming an incubation mixture comprising the compositions recited in element a);
c) incubating the mixture of step b) under conditions and for a period of time appropriate for Hh autoprocessing;
d) detecting fluorescence or luminescence in the incubation mixture of step c);
e) comparing the fluorescence or luminescence detected in step d) to fluorescence or luminescence detected using an otherwise identical incubation mixture which lacks the compound of element a) iii), a decrease in fluorescence or luminescence in step d) being indicative of the presence of a compound that inhibits autoprocessing of Hh.

2. The method of claim 1 where the nucleophile is a sterol.

3. The method of claim 1 where the nucleophile is cholesterol.

4. The method of claim 1 where the nucleophile is a thiol.

5. The method of claim 1 where fluorescent protein is selected from a group consisting of GFP, BFP, CFP and YFP and the red fluorescent protein from Discosoma and aequorin.

6. The method of claim 1 where luminescent protein is luciferase.

7. A method for the screening of compounds that block the autoprocessing of the Hedgehog protein in a cell-free system, said method comprising:
a) providing:
i) an expression vector encoding an N-terminal peptide-Hh fusion protein, said fusion protein comprising: (1) a Hh protein truncated at the N-terminal signaling domain; and (2) a fluorescently labeled small peptide fused in-frame with the Hh protein truncated at the N-terminal signaling domain; wherein cleavage of the fusion protein by Hh autoprocessing function results in a change in fluorescence polarization by the fluorescently labeled small peptide;
ii) a nucleophile initiator; and,
iii) a compound to be tested for the ability to inhibit Hedgehog autoprocessing;
b) forming an incubation mixture comprising the compositions recited in element a);
c) incubating the mixture of step b) under conditions and for a period of time appropriate for Hh autoprocessing;
d) detecting fluorescence polarization in the incubation mixture of step c);
e) comparing the fluorescence polarization in the mixture of step d) to the fluorescence polarization of an otherwise identical incubation mixture which lacks the compound of element a) iii), a change in fluorescence polarization of the mixture of step d) being indicative of the presence of a compound that inhibits autoprocessing of Hh.

8. The method of claim 7 wherein said fluorescently labeled small peptide contains the sequence Cys-Cys-X-X-Cys-Cys [SEQ ID NO.: 1], X being any amino acid residue, and said fluorescent label is applied by contact with a fluorescent biarsenical to form a fluorescent adduct.

9. The method of claim 8 where the sequence X-X is Gly-Pr.

10. The method of claim 8 where the fluorescent biarsenical is 4′,5′-bis(1,3,2-dithioarsolan-2-yl) fluorescein.

11. The method of claim 7 where the nucleophile is a sterol.

12. The method of claim 7 where the nucleophile is cholesterol.

13. The method of claim 7 where the nucleophile is a monothiol.

14. A cell-based method for the screening of compounds useful in the modulation of autoprocessing of the Hedgehog protein, said method comprising:
a) providing:
i) a test cell comprising: 1) a first expression vector comprising a nucleotide sequence encoding a functionally active, unprocessed Hh protein; and 2) a second expression vector comprising a nucleotide sequence encoding a luciferase gene in-frame with a Hh-responsive gene,
ii) a compound to be tested for the ability to inhibit Hedgehog autoprocessing, and;
b) forming an incubation mixture comprising the compositions recited in element a);
c) incubating the mixture of step b) under conditions and for a period of time appropriate for Hh autoprocessing;
d) detecting luminescence in the incubation mixture of step c);
e) comparing the luminescence of the incubation mixture of step d) to the luminescence of an otherwise identical incubation mixture which lacks the compound of element a) ii), a decrease in luminescence of the incubation mixture of step d) being indicative of the presence of a compound that inhibits autoprocessing of Hh.

15. The method of claim 14, wherein the Hh-responsive gene is selected from a group consisting of the Gli gene, the Patched gene, the N-Myc gene and the IGF2 gene.

16. A cell-based method for the screening of compounds useful in the inhibition of autoprocessing of the Hedgehog protein, said method comprising:
a) providing:
i) a test cell comprising: an expression vector, said expression vector comprising a nucleotide sequence encoding a functionally active, unprocessed Hh protein; and,
ii) a compound to be tested for the ability to inhibit Hedgehog autoprocessing, and;
b) forming an incubation mixture comprising the compositions recited in element a);
c) incubating the mixture of step b) under conditions and for a period of time appropriate for Hh autoprocessing;
d) detecting by Western Blotting the efficiency of the autoprocessing of the hedgehog protein;
e) comparing the efficiency of hedgehog autoprocessing to an otherwise identical incubation mixture which lacks the compound of element a) ii), wherein the detection of full-length hedgehog protein in the mixture of step d) is indicative of the presence of a compound that inhibits autoprocessing of Hh.

17. A cell-based method for the screening of compounds useful in the inhibition of autoprocessing of the Hedgehog protein, said method comprising:
a) providing:
i) a test cell comprising: an expression vector comprising a nucleotide sequence encoding a functionally active, unprocessed Hh protein; and,
ii) a compound to be tested for the ability to inhibit Hedgehog autoprocessing, and;
b) forming an incubation mixture comprising the compositions recited in element a);
c) incubating the mixture of step b) under conditions and for a period of time appropriate for Hh autoprocessing;
d) detecting by Mass Spectrometry the efficiency of the autoprocessing of the hedgehog protein;
e) comparing the efficiency of hedgehog autoprocessing to an otherwise identical incubation mixture which lacks the compound of element a) ii), wherein the detection of full-length hedgehog protein in the mixture of step d) is indicative of the presence of a compound that inhibits autoprocessing of Hh.

18. A cell-based method for the screening of compounds useful in the inhibition of autoprocessing of the Hedgehog protein, said method comprising:
a) providing:
i) a test cell comprising: 1) a first expression vector comprising a nucleotide sequence encoding a functionally active, unprocessed Hh protein; and 2) a second expression vector comprising a nucleotide sequence encoding a luciferase gene fused in-frame with a Hh-responsive gene,
ii) a control cell comprising: 1) a first expression vector, said first expression vector comprising a nucleotide sequence encoding a pre-processed Hh protein; and 2) a second expression vector, said second expression vector comprising a nucleotide sequence encoding a luciferase gene fused in-frame with a Hh-responsive gene,
iii) a compound to be tested for the ability to inhibit Hedgehog autoprocessing, and;
b) forming 1) a first incubation mixture comprising the compositions recited in elements a)i) and a)iii), and 2) a second incubation mixture comprising the compositions recited in elements a)ii) and a)iii), and;
c) incubating the first and second incubation mixtures of step b) under conditions and for a period of time appropriate for Hh autoprocessing;
d) detecting luminescence in the first and second incubation mixtures of step c);
e) comparing the luminescence of the first and second incubation mixtures, wherein a decrease in luminescence of the first incubation mixture as compared to the second incubation mixture being indicative of the presence of a compound that inhibits autoprocessing of Hh.

19. The method of claim 18, wherein the Hh-responsive gene is selected from a group consisting of the Gli gene, the Patched gene, the N-Myc gene and the IGF2 gene.

20. A composition effective in the inhibition of Hh autoprocessing, said composition comprises a compound selected from a group consisting of compound #4 of FIG. 3 and compound #3 of FIG. 7.
